(19) United States
(12) Patent Application Publication
(10) Pub. No.: US 2022/0365034 A1
(43) Pub. Date: Nov. 17, 2022

(54) NONDESTRUCTIVE ULTRASONIC ELASTOGRAPHIC IMAGING FOR EVALUATION OF MATERIALS

(71) Applicants: University of North Texas, Dallas, TX (US); ECHONOVUS INC., Denton, TX (US)

(72) Inventors: Arup NEOGI, Dallas, TX (US); Ezekiel WALKER, Denton, TX (US); Jin YUQI, Dallas, TX (US); Tae-Youl CHOI, Dallas, TX (US); Arkadii KROKHIN, Dallas, TX (US)

(21) Appl. No.: 17/612,193

(22) PCT Filed: May 22, 2020
(86) PCT No.: PCT/US2020/034351

§ 371 Date: Nov. 17, 2021

Related U.S. Application Data
(60) Provisional application No. 62/852,935, filed on May 24, 2019.

(51) Int. Cl. G01N 29/09; G01N 29/07; G01N 29/28; G01N 29/06; G01N 29/04

(52) U.S. Cl. CPC G01N 29/09; G01N 29/07; G01N 29/28; G01N 29/0645; G01N 29/043; A61B 8/587

(57) ABSTRACT

A method of non-destructive evaluation of mechanical properties of a material using ultrasonic waves in a monostatic configuration is disclosed. The method comprises remotely scanning a sample of the material without directly contacting the sample, measuring an acoustic impedance of the scanned sample, and calculating mechanical properties of the material using the acoustic impedance.

### CROSS-REFERENCE TO RELATED APPLICATIONS

The present application claims priority to U.S. Provisional Application No. 62/852,935 filed on May 24, 2019 and entitled “NONDESTRUCTIVE ULTRASONIC ELASTOGRAPHIC IMAGING FOR EVALUATION OF MATERIALS,” which is incorporated herein by reference in its entirety.

### STATEMENT REGARDING GOVERNMENTALLY SPONSORED RESEARCH OR DEVELOPMENT

The U.S. government has certain rights in the invention. This work is supported by an Emerging Frontiers in Research and Innovation grant from the National Science Foundation (Grant No. 1741677).

### BACKGROUND

Existing elastographic techniques for materials use an external force with physical contact being applied to the sample. This presents limitations of the current techniques in that that they require the need for an external pressure, which leads to difficulty in detecting deformation in stiff materials and the need for a bistatic setup for some methods.

### SUMMARY

In an embodiment, a method of non-destructive evaluation of mechanical properties of a material using ultrasonic waves in a monostatic configuration is disclosed. The method comprises remotely scanning a sample of the material without directly contacting the sample, measuring an acoustic impedance of the scanned sample, and calculating mechanical properties of the material using the acoustic impedance.

In another embodiment, a system for non-destructive evaluation of mechanical properties of a material using ultrasonic waves in a monostatic configuration is disclosed. The system comprises a transducer configured to remotely scan a sample of the material without directly contacting the sample. The system also comprises a computer having a configuration and programming instructions to measure an acoustic impedance of the scanned sample and calculate mechanical properties of the material using the acoustic impedance.

These and other features will be more clearly understood from the following detailed description taken in conjunction with the accompanying claims.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 is a diagram of a system for non-destructive evaluation of the mechanical properties of a material using ultrasonic waves in a monostatic configuration according to some embodiments.

FIG. 2 is a diagram of the relation of reflection in the nth material and emitted pulse in term of acoustic pressure according to some embodiments.

FIG. 3 is an acoustic pressure distribution from transducer emitted pulse to the second echo which passed into sample then reflected according to some embodiments.

FIG. 4 is a graphic representation of ratio difference between sample and reference material acoustic impedance value dependent effective coefficient for p₁(t) according to some embodiments.

FIG. 5A is an illustration of a scanned image of a hard and soft material composite sample according to an embodiment.

FIG. 5B is an illustration of traditional A-Mode imaging logarithmically scaled according to an embodiment.

FIG. 5C is an illustration of traditional A-Mode imaging in linear scale according to an embodiment.

FIG. 6A is an illustration of a scanned image of the hard and soft material composite sample according to an embodiment.

FIG. 6B is an illustration of the EBME mapping of the bulk modulus (K) from Eq. 10 according to an embodiment.

FIG. 6C is an illustration of the EBME effective density mapping from Eq. 9 according to an embodiment.

FIG. 7A is an illustration of a scanned image of the hard and soft material composite sample according to an embodiment.

FIG. 7B is an illustration of the EBME logarithmic scale intensity of FIG. 6B according to an embodiment.

FIG. 7C is an illustration of the EBME bulk modulus in a logarithmic scale according to an embodiment.

FIG. 8A is an illustration of a scanned image of a hard material composite sample according to an embodiment.

FIG. 8B is an illustration of traditional A-Mode imaging logarithmically scaled according to an embodiment.

FIG. 8C is an illustration of traditional A-Mode imaging in linear scale according to an embodiment.

FIG. 9A is an illustration of a scanned image of the hard material composite sample according to an embodiment.

FIG. 9B is an illustration of the Effective Bulk Modulus Elastography (EBME) mapping of the bulk modulus (K) from Eq. 10 according to an embodiment.

FIG. 9C is an illustration of the EBME effective density mapping from Eq. 9 according to an embodiment.

FIG. 10A is an illustration of a scanned image of the hard material composite sample according to an embodiment.

FIG. 10B is an illustration A Mode scanned imaging in logarithmic scale according to an embodiment.

FIG. 10C is an illustration of the EBME bulk modulus in a logarithmic scale according to an embodiment.

FIG. 11A is an illustration of a scanned image of a first sample of soft tissue phantom composite according to an embodiment.

FIG. 11B is an illustration of traditional A-Mode imaging logarithmically scaled according to an embodiment.

FIG. 11C is an illustration of traditional A-Mode imaging in linear scale according to an embodiment.

FIG. 12A is an illustration of a scanned image of the first sample of soft tissue phantom composite according to an embodiment.

FIG. 12B is an illustration of the EBME mapping of the bulk modulus (K) from Eq. 10 according to an embodiment.

FIG. 12C is an illustration of the EBME effective density mapping from Eq. 9 according to an embodiment.

FIG. 13A is an illustration of a scanned image of the first sample of soft tissue phantom composite according to an embodiment.

FIG. 13B is an illustration of a relative EBME derived bulk modulus ratio as compared to water according to an embodiment.

FIG. 13C is an illustration of a relative density of the first sample as compared to water according to an embodiment.

FIG. 14A is an illustration of a scanned image of a second sample of soft tissue phantom composite according to an embodiment.

FIG. 14B is an illustration of traditional A-Mode imaging logarithmically scaled according to an embodiment.

FIG. 14C is an illustration of traditional A-Mode imaging in linear scale according to an embodiment.

FIG. 15A is an illustration of a scanned image of the second sample of soft tissue phantom composite according to an embodiment.

FIG. 15B is an illustration of relative bulk modulus scaled to water according to an embodiment.

FIG. 15C is an illustration of relative density scaled to water according to an embodiment.

FIG. 16 is a diagram of a system for non-destructive evaluation of the mechanical properties of a material using ultrasonic waves in a monostatic configuration according to some embodiments.

### DESCRIPTION

Ultrasound technologies are continuously developing and enjoy broad usage in biomedical and manufacturing engineering applications for sensors, cleaning, welding, material characterization, and imaging. In manufacturing engineering, ultrasonic characterization is an alternative test method for mechanical properties that is faster than tensile and compression tests on stiff, isotropic materials such as metals and alloys. By utilizing both the measured longitudinal and transverse speed of sound, the sample thickness, and material density, mechanical properties such as the Young's modulus, shear modulus, and Poisson's ratio can be extrapolated from the method. The method, however, is commonly insufficient for soft materials like biological tissues due to the effects of dispersion and attenuation.

Ultrasonic imaging, which is widely used across many disciplines in both academic and industrial settings, incorporates ultrasonic characterization techniques for visualization. Basic ultrasonic imaging (B mode imaging) is based on time of flight measurement of the ultrasound pulse, and is often displayed as a greyscale map where intensities are connected to an elastic property. Conversely, A mode imaging measures energy level (amplitude) of reflected waves at a fixed distance. Resolution of the imaging modes are closely related to wave frequency for the axial direction, and beam waist size for the lateral direction with frequencies between 1 to 20 MHz most often used. Higher frequency devices are used to improve imaging resolution with frequencies as high as 100 MHz or even hypersound. For ultrasonic attenuation, measures of the ratio of the attenuation coefficient between a sample phantom and reference phantom are collected and mapped in color grades to provide in depth detail of the features of a sample.

Ultrasound elastography is more commonly used in biomedical applications, with elastographic imaging (M mode imaging) a particular focus of recent research. Strain map elastography, one of the earlier developed methods in M mode imaging, is dependent on compressive changes in a composite sample's thickness. Compressive stress on materials with differing elastic properties causes varying degrees of deformation in the linear elastic range. Strain mapping utilizes the change in time of flight information between a sample with and without stress to reconstruct the change in thickness of composites within the sample and derive the Young's modulus and Poisson ratio. The earliest designs of strain elastography applied compressive force on a sample manually using an ultrasonic transducer, but further developed to be an automatic applied and held force. In practicality, ultrasound elastography is ineffective when a target material is hard, deep, and/or has fluid in interstitial spacings. Additionally, though this method does not offer quantitative values for the bulk and Young's modulus, it can distinguish materials which have distinct Poisson's ratios in real time.

Techniques that use acoustic radiational force instead of mechanical forces to determine elastic properties fall under impulse strain imaging. From multiple measurements of displacement information with acoustic radiational force, small deformation differences can be found and used for calculating the Young's modulus. Elasticity information is usually represented on a color scale with hard, or stiff, features represented by warm tones and soft, more malleable features by cold tones.

Impulse strain mapping and strain map elastography are commonly used methods in both laboratory and commercial settings. Poisson's ratio mapping, however, is a technique looking for vertical strain information similar to strain and impulse strain imaging, but is restricted to laboratory use. For the method, the sample is in water ambient, and a vertical compressional force on the sample raises the water level in the tank. The measured horizontal elongation provides effective Poisson's ratio map calculated on a scale from 0 to 0.5. In commercial devices, strain elastography or impulse strain mapping are usually used, with either the absolute or relative elastic values represented in a color scale overlapped on grey scale B-mode images.

Another popular M-mode imaging modality is Shear Wave Elasticity Imaging (SWEI) and is comprised of three methods: Point Shear Wave (PSW) imaging, Surface Shear Wave (SSW) imaging, and Transient Shear Wave (TSW) imaging. Whereas strain elastography or impulse strain mapping primarily are determined with longitudinal waves, SWEI techniques require shear waves. SSW imaging, a relatively recent SWEI method, measures shear wave dispersion and velocity in the temporal domain. The method uses a bistatic setup to provide one focal surface by two overlapped focal zones and combines the overlapped focal zones with an external radiation force to obtain the non-quantitative shear elasticity imaging. PSW and TSW differ from SSW in that they on require a monostatic setup.

Point shear wave imaging utilizes shear waves and is used to find either the Young's or shear modulus (elasticity) by measuring the change of the shear wave propagation speed in a focal zone with an applied radiational compressive force, lateral force, or shear force. SWEI is dependent on adequate sample deformation to modify to the measured shear wave speed of sound. For point shear applications, SWEI provides accurate results when used in samples greater than 20 mm thick or harder tissue materials such as muscle.

In the lower frequency range, Supersonic Shear Imaging (SSI), also called Transient Shear imaging, is setup using a monostatic ultrasound probe connected to an external acoustic radiational force generator. An ultrasound probe continuously records B mode images to find deformation in a specified temporal range after the shear force propagates in the sample. The deformation information forms the basis of a quantitative Young's modulus map. Shear wave velocity and temporal dispersion, represented quantitatively by echo phase shift due to an external dynamic force, is also used for transient shear imaging in inhomogeneous tissues using a monostatic arrangement. Drawbacks of SWEI in biomedical applications are primarily functions of the impact of body fluids on elastographic results due to the inability of fluid to transmit shear waves.

Techniques to image the elastic properties of materials have been primarily geared towards soft, biological samples for biomedical applications, whereas for hard materials the techniques have been used primarily in non-imaging modalities. Effective stiffness (bulk modulus) of monoatomic metal and alloys undergoes significant cavitation erosion due to environmental impact. As noted above, the limitations of current techniques include the need for an external pressure, difficulty in detecting deformation in stiff materials, and the need for a bistatic setup for some methods.

Disclosed herein are systems and methods for non-destructive evaluation of the mechanical properties of a material using ultrasonic waves in a monostatic configuration, and in particular methods and devices using strain map imaging and shear wave elastography to remotely probe a sample with direct contact and estimate the bulk or Youngs modulus of the material from the reflection of ultrasonic waves. Herein Effective Bulk Modulus Elastography (EBME) imaging is presented as a new imaging technique. From measuring the acoustic impedance of the scanned sample, effective bulk modulus and effective density mapping can be constructed using classical speed of sound theories K=ρc²=Z_{c} and ρ=Zc⁻¹. The method can provide effective stiffness information with bulk modulus scale elastography using a monostatic setup with longitudinal pulses absent any external radiational or cyclidic stress application on the sample. Derivation of the method is given below.

The Elastic Bulk Modulus Elastography (EBME) method disclosed herein does not require an external, forced deformation of the material being analyzed, and is functional for both hard and soft materials. The technique does not require explicit knowledge of the elastic properties of a reference material for application. However, the sensitivity of the detection equipment should be adequately known for EBME effectiveness. As will be illustrated through the description and examples herein, EBME proved effective in discerning between tissue phantoms with small variations in their bulk elastic properties in addition to accurately determining the density and bulk modulus of hard materials. Use of the relative density and bulk modulus of a material using EBME may enable faster detection of unwanted defects in a material. As demonstrated herein, the errors in EBME may be primarily a function of low signal to noise ratios from impedance matched tissue phantoms that have low reflectivity in water ambient.

The elastography technique presented herein is unique as compared with other existing elastography methods. Table 1, reproduced below, is a comparison of the existing ultrasonic elastography methods and the elastography technique presented herein.

TABLE 1
ULTRASONIC ELASTOGRAPHY METHODS COMPARISON

| | Wave | External | Elasticity | Water | Input |
|---|---|---|---|---|---|
| Methods | Mode | force | values | ambient | Values |
| Strain Map | L | ✓ | X | X | X |
| [14, 15, 17] | | | | | |
| Impulse | L or T | ✓ | E | X | Z, f_{σ}, σ |
| strain map | | | | | |
| [18, 19] | | | | | |
| Poisson' | L | ✓ | ν | ✓ | X |
| ratio map | | | | | |
| [13, 20] | | | | | |
| Transient | T | ✓ | E | X | ρ, σ, f_{σ}, d |
| shear wave | | | | | |
| map | | | | | |
| [29] | | | | | |
| Point shear | T | ✓ | E or G | X | ρ, σ, f_{σ}, d |
| wave map | | | | | |
| [23, 24, 25] | | | | | |
| Surface | T | ✓ | E or G | X | ρ, σ, f_{σ}, d |
| shear wave | | | | | |
| map | | | | | |
| [22] | | | | | |
| EBME | L | X | K | ✓ | d, S, Z₀ |
| Bulk | | | | | |
| Modulus | | | | | |
| EBME | L | X | ρ | ✓ | d, S, Z₀ |
| Density | | | | | |

All existing methods need an external force or sonic pulse induced deformation of the sample, unlike EBME. Deformations of a sample may incur unwanted peripheral effects. As with other methods, some knowledge of at least the ambient materials should be known for EBME to be effective. Standard practice for prior methods is the knowledge or assumption of a reference bulk modulus or density for creating elastography maps. In EBME, direct knowledge of the bulk modulus or density of a reference material is not explicitly required. However, the pressure-voltage sensitivity of the equipment, which can be standardized to a medium, may be used for EBME effectiveness.

The need to deform a medium for gathering elastographic information make characterizations of hard materials more difficult. Metals or hard plastic, specifically, do not easily deform without significant applied pressure due to large elasticity constants. Moreover, the linear elasticity strain range is normally small for metals and alloys, and especially for hard plastic. Strain map and Poisson's ratio map might not be capable for these materials because of non-ultrasonic measurable small linear range strain. Large stresses can easily exceed the small linear deformation range. Polymer plastics, many alloys, even polycrystalline metals are usually non-isotropic materials, and their anisotropy can be measured by shear waves. Once the results from shear wave maps become directional, those shear wave methods my not be reliable anymore in imaging. EBME, as demonstrated in the experiments below, maintains an advantage over existing techniques in that it is effective for both hard and soft materials. The lack of a required external, deforming pressure also means EBME is a form of remote sensing that can readily be transportable.

As will be discussed further below, additional signal processing to improve the SNR of the collected data may improve accuracy of EBME method. Additionally, analysis was undertaken with the premise of exclusive use of longitudinal waves when the ambient medium comprises a liquid. In some embodiments, transverse waves alone or in combination with longitudinal waves may be used. Further considerations of transverse waves and their impact on the efficacy of EBME may improve effectiveness of the method as applied to hard and soft materials.

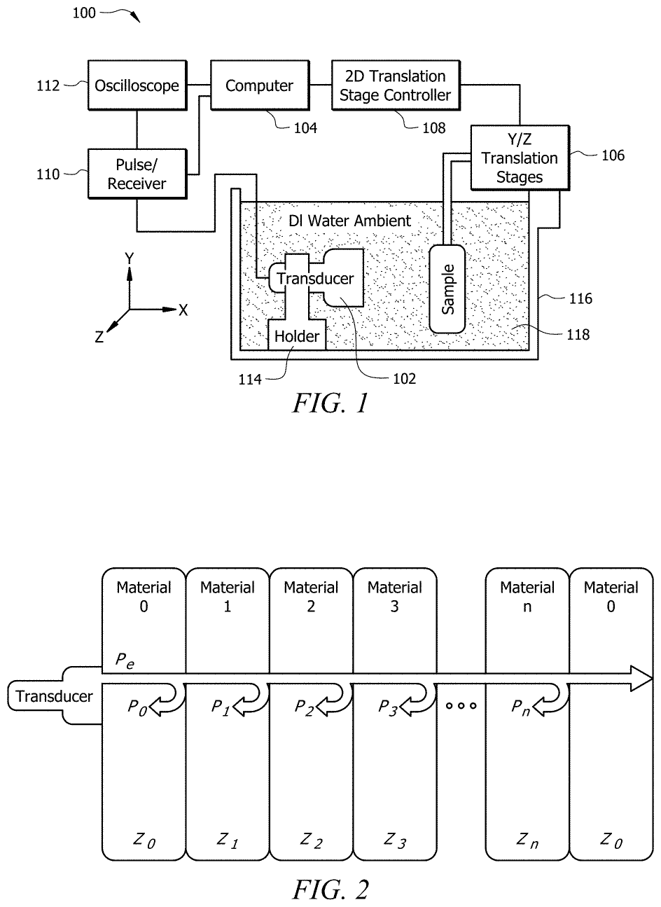

Turning to FIG. 1, a diagram of a system 100 for non-destructive evaluation of the mechanical properties of a material using ultrasonic waves in a monostatic configuration is illustrated. In an embodiment, raster scanned imaging was completed using a computer controlled, automated experimental system as shown in FIG. 1. The system 100 may comprise a transducer 102 and a computer 104. In an embodiment, the transducer 102 can comprise an unfocused immersion transducer. The computer 104 may have a configuration and programming instructions to perform various steps such as measure an acoustic impedance of the scanned sample and calculate mechanical properties of the material using the acoustic impedance. The system 100 may also comprise Y/Z translation stages 106 and 2D translation stage controller 108. The system 100 may further comprise a pulse/receiver 110 connected to the transducer 102, and an oscilloscope 112 to acquire data. In an embodiment, the system 100 further comprises a holder 114 and the transducer 102 is mounted to the holder 114. In such an embodiment, the holder 114 may be mounted with a container 116 filled with an ambient medium 118.

The waveform and frequency spectrum may be recorded for a period of time between 1 second and a minute at each raster scan location interval on the y-axis and the same or a different interval on the vertical, z-axis. In an embodiment, the ambient medium consisted of a liquid such as water for more efficient signal generation and detection from the immersion transducer. The collected data was then post-processed to create elasticity maps.

An analytical model is used to determine the properties of the material being tested. To determine the impedance Z of the scanned sample, a model can be used to determine the intensity and acoustic pressure relation equation in a medium,

$${I}_{n} ( x , t ) = \frac{1}{{Z}_{n}} \cdot \frac{1}{{t}_{f} - {t}_{i}} {\int }_{{t}_{i}}^{{t}_{f}} {{p}_{n} ( x , t )}^{2} d t \qquad ( Eq . 1 )$$

Where Iₙ and pₙ are the acoustic intensity and acoustic pressure of the n^{th} pulse respectively, and Zₙ, the acoustic impedance of the n^{th} material. The variables t_{i,n} and t_{f,n} are defined to be the starting and ending time of the n^{th} pulse envelope, where the pulse is for material Zₙ. In the time domain, tᵢ and t_{f} can be found algorithmically.

The beginning of a pulse envelope, tᵢ, can be calculated by examining the transient data from the end of the prior pulse and set as the point when a continuous time equal to half the pulse width in the ambient material had more than 110% of the maximum noise level amplitude in that time window. In some embodiments, the ambient material can be water with a pulse width of 1 μs. The result tᵢ can be set as the point when a continuous 0.05 μs exceeded 110% of the maximum noise level. The end of a pulse envelope, t_{f}, can be calculated by examining the transient data from tᵢ and set as the point when a continuous, following half pulse width of data (0.5 μs) has less than 90% of the maximum noise level in that time window.

Since all detection happens at the stationary transducer, we neglect the explicit terms for x in p(x, t) and I(x, t) as x is the same for all emitted and detected pulses. We first define pₑ(t), to be the initial, emitted pulse, and pₙ(t) to be the subsequent n^{th} reflections, where p₀(t) is the reflection from the first boundary as shown in FIG. 2. The intensity of a reflected pulse can be defined as yₙ(t) and transformed to the frequency domain Yₙ(f) by Fourier transformation. The intensity of this pulse can be written as a sum of power of all frequency components,

Iₙ=Σ_{k=f}_{₁}^{N}Yₙ(f)k (Eq. 2)
Where N is the number of discretized frequency components between f₁ and f₂ from Fourier transformation. In some embodiments, N can be 241, and the upper and lower bound of the transducer can be restricted to f₁=0.2 MHz and f₂=0.8 MHz due to its sensitivity.

The relation between acoustic pressure of emitted pulse from transducer and the reflected pulse from the further interface of each material layers back to transducer can be expressed as

p₁(t)=(pₑ(t)−p₀(t))·r_{1,2}·t_{1,0}

p₂(t)=(pₑ(t)−p₀(t))·t_{1,2}·r_{2,3}·t_{2,1}·t_{1,0}

p₃(t)=(pₑ(t)−p₀(t))·t_{1,2}·t_{2,3}·r_{3,4}·t_{3,2}·t_{2,1}·t_{1,0}

[symbol]

pₙ(t)=(pₑ(t)−p₀(t))·(Πᵢ₌₂ⁿt_{n-1,n})·r_{n,n+1}·(Πᵢ₌₁ⁿt_{n,n-1}) (Eq. 3)

Where t_{n-1,n} is a transmission coefficient when the wave propagates from material n−1 to material n, and can expressed as t_{n-1,n}=(2Zₙ)/(Zₙ₋₁+Zₙ). r_{n,n+1} is the reflection coefficient at the interface between material n and n+1 when the wave propagates from material n. t_{n,n-1} is the transmission coefficient of acoustic pressure from material n to material n−1, opposite direction of t_{n-1,n}. This coefficient can be expressed as t_{n-1,n}=(2Zₙ₋₁)/(Zₙ₋₁+Zₙ). pₙ is the detected pressure, and is the reflection from the furthest boundary of material n that eventually is detected by the transducer.

In Eq. 3, pₑ(t)−p₀(t) is acoustic pressure transmitted into material 1. Πᵢ₌₂ⁿt_{n-1,n} is the product of all transmission coefficients from material 1 to material n. As part of the acoustic pressure is reflected at interface between material n and material n+1, Πᵢ₌₁ⁿt_{n,n-1} is the product of all transmission coefficients of the returning acoustic pressure from material n to transducer.

Besides measurements of pₑ(t) and p₀(t), from each recorded echo p₁(t), p₂(t) p₃(t), . . . pₙ(t), Eq. 1, 2, and 3 can be used to find the effective propagation coefficient, ξₙ, of each echo as a function of pₑ(t)−p₀(t),

$$1. {ξ}_{1} = \frac{{\int }_{{t}_{i}}^{{t}_{f}} {( {p}_{e} ( t ) - {p}_{0} ( t + τ ) )}^{2} d t}{( {t}_{f} - {t}_{i} ) {\sum }_{k = {f}_{1}}^{N} {{Y}_{1} ( f )}_{k}} {ξ}_{2} = \frac{{\int }_{{t}_{i}}^{{t}_{f}} {( {p}_{e} ( t ) - {p}_{0} ( t + τ ) )}^{2} d t}{( {t}_{f} - {t}_{i} ) {\sum }_{k = {f}_{1}}^{N} {{Y}_{2} ( f )}_{k}} \begin{matrix}{ξ}_{3} = \frac{{\int }_{{t}_{i}}^{{t}_{f}} {( {p}_{e} ( t ) - {p}_{0} ( t + τ ) )}^{2} d t}{( {t}_{f} - {t}_{i} ) {\sum }_{k = {f}_{1}}^{N} {{Y}_{3} ( f )}_{k}} \\ ⋮\end{matrix} {ξ}_{n} = \frac{{\int }_{{t}_{i}}^{{t}_{f}} {( {p}_{e} ( t ) - {p}_{0} ( t + τ ) )}^{2} d t}{( {t}_{f} - {t}_{i} ) {\sum }_{k = {f}_{1}}^{N} {{Y}_{n} ( f )}_{k}} \qquad ( Eq . 4 )$$

Where τ is temporal difference between pₑ(t) and p₀(t), and Σ_{k=f}_{₁}^{N}Yₙ(f)ₖ is the intensity of the n^{th} reflected pulse, written as a sum of the power of all frequency components from the Fourier transformed waveform of pₙ(t).

The effective propagation coefficient ξₙ of the reflections are also defined as ξ₁=Z₁·(r_{1,2}·t_{1,0})², ξ₂=Z₂·(t_{1,2}·r_{2,3}·t_{2,1}·t_{1,0})², and ξₙ=Zₙ·((Πᵢ₌₂ⁿt_{n-1,n})·r_{n,n+1}·(Πᵢ₌₁ⁿt_{n,n-1}))². With known acoustic impedance of ambient material Z₀, the acoustic impedance values of the sample materials Z₁, Z₂, Z₃, . . . Zₙ can be solved recursively, and the subsequent density and bulk modulus values can be determined using equations ρ=Z/c and K=ρc²=Zc.

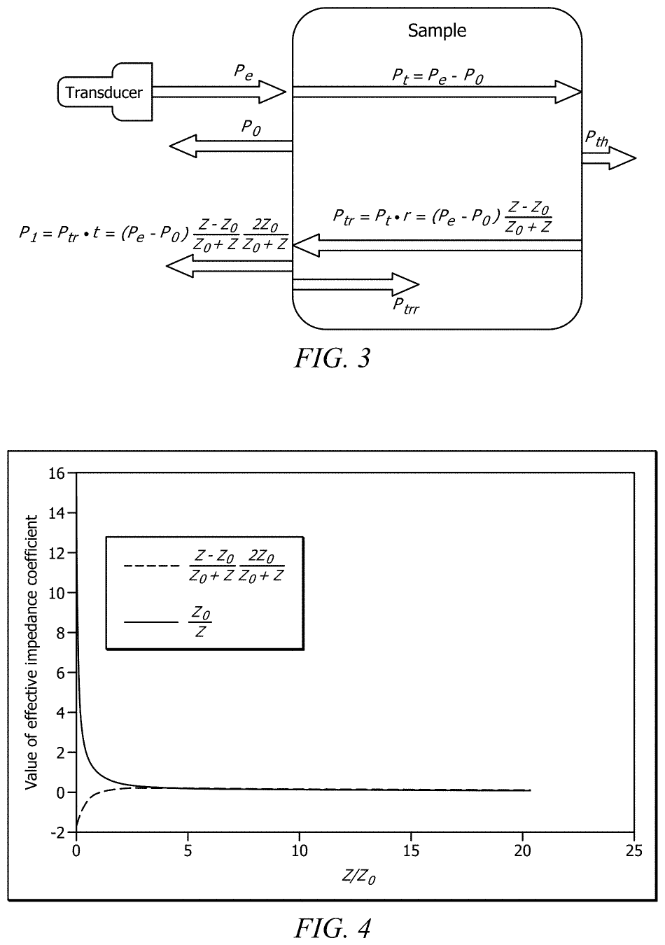

FIG. 3 exhibits the acoustic pressure distribution of a simplified case as an example where there is only one material in addition to the ambient. The acoustic pressure of second echo (the reflected energy which has transmitted into sample) p₂ is defined as

$${p}_{1} ( t ) = ( {p}_{e} ( t ) - {p}_{0} ( t ) ) \frac{Z - {Z}_{0}}{Z + {Z}_{0}} \frac{2 {Z}_{0}}{Z + {Z}_{0}} \qquad ( Eq . 5 )$$

Where pₑ(t) is emission acoustic pressure from transducer, and p₀(t) is acoustic pressure of the echo reflected back from the front interface of ambient and sample material. r is a reflection coefficient r=(Z−Z₀)/(Z+Z₀) which describes the ratio of the energy reflected from back interface between scanned sample and water, and t is a transmission coefficient t=2Z₀/(Z+Z₀) which describes the ratio of the energy transmitted from front interface between scanned sample and water going back to transducer. Z is acoustic impedance of the scanned sample, and Z₀ is acoustic impedance of the ambient material, defined as Z₀=ρ₀c₀. Here, water is used as the ambient material resulting in ρ₀ and c₀ being the density and speed of sound in water respectively.

For determining the sample impedance, we introduce an estimated replacement coefficient

$$\frac{{Z}_{0}}{α Z}$$

in Eq. 6 as a simplification factor for Eq. 5 so Z can be presented explicitly as a function of the acoustic impedance of the ambient material, and the detected pulses.

$${p}_{1} ( t ) = \frac{{Z}_{0} ( {p}_{e} ( t ) - {p}_{0} ( t ) )}{a Z} \qquad ( Eq . 6 )$$

Where α is a scaling coefficient. For this simplified case of a single material in the ambient medium, FIG. 4 shows the relative behavior of Eq. 5 and Eq. 6 as a function of the ratio of the unknown sample impedance and that of the ambient. For high impedance mismatch between the two media, α asymptotically approaches 1, and the simplified case replicates the ideal case well. As the impedance ratio between the ambient and analyzed materials approaches zero, the behavior of the simplified and ideal cases diverge. For an ambient of water, α approaches 2 in soft materials, specifically for tissue phantoms, and 1 for hard materials such as metals.

The practical implementation of the EBME technique requires knowledge of the sensitivity of the detector. In some embodiments, p₀ and p₁ can be obtained from the time dependent pulse voltage amplitude V₀(t) and V₁(t). p₀(t)=V₀(t)·S and p₁(t)=V₁(t)·S, where S is pressure-voltage sensitivity coefficient of the detector in units of

$$\frac{P a}{V} .$$

When combined with Eq. 1, the intensity becomes

$$I = \frac{1}{Z} \cdot \frac{1}{{t}_{f} - {t}_{i}} {\int }_{{t}_{i}}^{{t}_{f}} {( \frac{{Z}_{0} {p}_{e} ( t )}{a Z} - \frac{{Z}_{0} {p}_{0} ( t + τ )}{a Z} )}^{2} dt = \frac{{Z}_{0}^{2}}{{Z}^{3}} \cdot \frac{1}{{t}_{f} - {t}_{i}} {\int }_{{t}_{i}}^{{t}_{f}} {( {V}_{e} ( t ) \cdot S - {V}_{0} ( t + τ ) \cdot S )}^{2} d t \qquad ( Eq . 7 )$$

Where τ is the time delay between the starting point of the emission pulse and the first echo. Using Eq. 2 and Eq. 7, the acoustic impedance of the sample is

$$Z = {( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( Eq . 8 )$$

Combining Eq. 8 with the speed of sound of the sample determined from time of flight information and sample thickness, the density and bulk modulus then become

$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3} \qquad ( Eq . 9 )$$

$$K = {c ( \frac{{Z}_{0}^{2} {\int }_{{t}_{i}}^{{t}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3} \qquad ( Eq . 10 )$$

The model then allows for the determination of elastographic information of various materials. In some embodiments, the system can be used to determine the elastographic information of soft materials such a biological tissues. Existing research shows measured mechanical properties and acoustic properties of tissue using ultrasound can occupy a range of values instead. Organic tissues normally possess much larger acoustic impedance than water. As described in the experimental section, 6.8% gelatin tissue phantom can be representative of very soft liver tissue. Samples of 10% and 16.8% gelatin tissue phantom can be representative of tumors at different stages. Prior work on the difference of speed of sound between healthy tissue and tumor tissue has been measured at less than 2%. The density and bulk modulus of tumor tissue, however, has been found to be discernably larger than healthy tissue. The EBM technique as described herein for the remote determining of the bulk modulus and density map can allow for differences between tissue phantom equivalents of tumorous and healthy tissue to be determined. For medical applications, EBME may provide a path to practical biomedical elastography and tomography applications.

The physical properties of healthy and tumor tissues vary amongst and between states of health of patients. Additionally, the accurate determination of elastic properties using a monostatic setup requires adequate pressure wave reflection which presents challenges for some liquids in soft materials. Elastography maps that use absolute values for the bulk modulus, density, and any other elastic properties may not be the most effective modality for examination of a material. Relative density (ρᵣ) and relative bulk modulus (Kᵣ) can potentially provide greater insight into the examination of a material. Setting the scale according to a fixed density or bulk modulus that is preferred, the user of EBME can qualitatively determine the elastic properties of a sample (e.g., as shown in FIGS. 12A-12C as discussed in more detail herein). For medical applications, this may be interpreted as setting the relative scales to the values of a known healthy tissue in an analysis, giving the trained eye more efficient guidance to areas of a sample that may be of concern.

The system can also allow heterogeneities to be accurately imaged and their material properties accurately characterized. The system may further have a large number of applications such as in medical imaging, medical diagnosis and medical therapeutic as well as in the characterization of material rheological properties.

The present systems and methods can also extend to a method of imaging inclusions within a material. A method according to an embodiment of the present invention broadly comprises the steps of i) inducing vibrations into a sample, ii) extracting the resonance frequencies from obtained echo/displacement spectra, and iii) imaging the shape of the variations within the sample.

Vibration of the sample can include forced mechanical perturbation of the sample using sonic or ultrasonic transducers. The transducers may induce longitudinal or transverse perturbation by physical contact with a sample. Physical contact with the sample may be achieved direct or indirect contact with the sample, only requiring an ambient medium that can support either longitudinal, transverse, or combined polarized sonic or ultrasonic waves. The acoustic impedance mismatch may be limited so that it does not exceed 0.999 between the ambient medium and the examined sample. In addition to longitudinal and transverse wave polarizations, other polarizations can also be used including, but not limited to, spherical, circular, conical and perpendicular antisymmetric. For substantially spherical samples, this may be achieved by the interaction of a torsional shear wave with the sample.

For biological applications, the frequency of the incident wave can range between a 0.1 hertz and 3.5 GHz depending on the size and material properties of the sample, for example between 50 and 1000 Hz. For the measurement of viscoelasticity of a sample within a container body, the frequency range may typically be between 1 Hz and 20 MHz depending on the precision of vibrations measurement technique, for example between 10 Hz and 20 kHz.

In biological applications, samples of a size between 1 mm and 10 m in diameter, for example between 5 mm and 20 mm, can be detectable by the present system and method. In industrial applications for viscoelasticity measurements for example, samples of a diameter between 0.1 mm and 40 m, for example between 5 mm and 30 mm may be assessed by the present system and method.

The embodiments having been generally described, the following examples are given as particular embodiments of the disclosure and to demonstrate the practice and advantages thereof. It is understood that the examples are given by way of illustration and are not intended to limit the specification or the claims in any manner.

### EXAMPLES

Three experiments were performed to evaluate the performance of this new method and are discussed below. In Experiment 1, a hard and soft material combined as a single sample were imaged. The hard, low dispersion material was an aluminum slab with a well-defined, large, rectangular area filled with the soft, dispersive material, silicone rubber. For Experiment 2, a series of hard materials in parallel were evaluated using the EBME technique to examine the effectiveness of EBME for distinguishing hard materials monostatically without an external applied stress. The series of hard materials consisted of copper, PVC plastic, and aluminum. As constructed in this work, EBME showed the greatest performance with hard materials due to their low dispersion and high impedance mismatch with the ambient. Experiment 3 pertained to application of EBME to soft, tissue-like materials and the capability of EBME to distinguish between soft materials that mimic healthy tissues and calcified or hardened tissues that may indicate ailments. The experiment contrasts three tissue phantoms synthesized using standard formulations for healthy and tumor-like tissue where elastic stiffness values are similar.

A system as described with respect to FIG. 1 was used in the experiments. For the experiments, a raster scanned imaging was completed using a MATLAB® controlled, automated experimental system. The transducer 102 was a single Olympus Panametrics V301 1″ 0.5 MHz unfocused immersion transducer. The system also included Y/Z translation stages 106 and 2D translation stage controller 108. For example, two Newport UE41PP stepper motor translation stages were connected and moved along the lateral (y-) and vertical (z-) axes with an attached sample using a Universal Motion Controller/Driver Model ESP300. The system 100 further included a pulse/receiver 110 connected to the transducer 102, and an oscilloscope 112 to acquire data. The pulse/receiver 110 comprises a JSR Ultrasonics DPR 300 Pulser/Receiver and the oscilloscope 112 comprises a Tektronix MDO 3024b oscilloscope. The waveform and frequency spectrum were recorded for 20 s at each raster scan location at 2 mm intervals on the y-axis and 1 mm intervals on the vertical, z-axis. In an embodiment, the ambient medium consisted of DI water for more efficient signal generation and detection from the immersion transducer. For all of the examples discussed below, all samples and materials that comprised the samples were at least the transducer 102 width to ensure they would be detectable by the unfocused transducer 102. The collected data was then post-processed to create elasticity maps.

### Sample Fabrication

For Experiment 1, an 8 mm width defect was cut in the center of a 7 mm thick, rectangular aluminum alloy 6061 slab using vertical milling machine Enco 1001586. Ecoflex 00-05 platinum cure silicone rubber component A and B was mixed in a 1:1 volume ratio for 10 minutes, then placed under vacuum for 10 minutes to degas. The liquid mixture was then filled into the slab defect and allowed to cure for two hours. For Experiment 2, low impurity copper, Jinan Jinbao Plastic Co., ltd solid PVC plastic block and aluminum block samples of 30 mm width, 35 mm width, and 35 mm width were placed side by side. The entire sample area consisting of the three metals was scanned in a single run. For Experiment 3, six tissue phantoms were created based on the synthesis process in Ultrasound Phantom Material,” J. Clin. Ultrasound, vol. 23, pp. 271-273, 1995. Samples containing 8.7 g, 17.0 g, 25.0 g, 42.0 g, 45.0 g and 56.0 g of Knox sugar free gelatin powder were mixed with 250 mL of boiling water and magnetically stirred for 15 minutes. The solution was then allowed to naturally cool to room temperature around 20° C. to minimize air bubbles in the final, solidified samples. The samples were then refrigerated at 5° C. for two days before analysis.

### Experiment 1—Hard and Soft Material Composite Sample

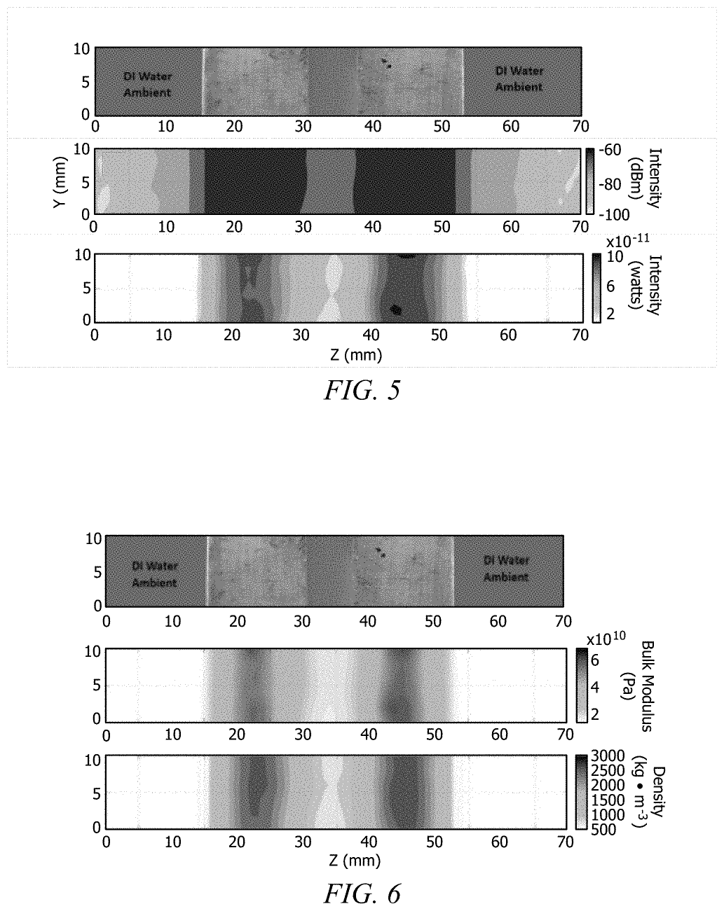

Experiment one concerns the application of the EBME technique to a sample comprised of both a hard material and a soft material. The scanned sample was a 38 mm wide aluminum slab with an 8 mm wide rectangular defect filled with silicone rubber as shown in FIGS. 5A, 6A, and 7A. Data was collected from a 2D, 70×10 mm (lateral×vertical) raster scan in deionized (DI) water ambient. FIG. 5B and FIG. 5C represent the intensity data in the logarithmic (5B) and linear (5C) scales. As FIG. 5B shows, A-Mode imaging in logarithmic scale clearly shows the silicone rubber filled rectangular defect between two sides of aluminum. The average width of the silicone rubber is around 9 mm and average aluminum width of each sides are both around 13 mm in the imaging.

Due to the size of transducer and the lateral beam width of the unfocused transducer, large diffraction patterns appear close to both sides of the aluminum. Water ambient exhibits similar contrast to the silicon as is to be expected due to the high impedance mismatch with aluminum, but relatively low impedance mismatch with silicon (FIG. 5B).

Linear scale A-Mode imaging is rarely utilized in practice. However, in the linear scale, resolution of the object decreases whereas the contrast increases greatly (FIG. 5C). The intensity gradients in the linear scale, resultant, again, from the transducer size and lateral beam width, make determination of the material boundaries tenuous at best. In comparison with FIG. 5B, the contrast between water, aluminum, and silicone increases greatly which has the potential to aid in the identification of heterostructures. Average width of the scanned sample is around 39 mm in linear scale, within 3% of the actual width.

As part of EBME, the bulk modulus and density can be extrapolated from acquired data using Eq. 9 and Eq. 10. Since the method is functionally dependent on the impedance of the ambient medium, and the accurate characterization of the pressure-voltage sensitivity of the detector, the bulk modulus and density are more correctly termed as effective bulk modulus and effective density. Quantitatively correct bulk modulus and density from EBME can be derived when the ambient medium and equipment characterization are accounted for accurately.

For Experiment 1, the effective bulk modulus and density are given in FIGS. 6B and 6C. As both the bulk modulus and density are on a linear scale, the impact of the low lateral resolution of the setup becomes apparent. The material boundaries are not well defined due to the gradient values at the transitions between materials. However, as with the intensity, the contrast in the elastic properties between the materials is very clearly seen in the contrasting values, especially at the center of a particular material.

In this setup, the bulk modulus and density values of aluminum are found to be 63.4 GPa and 2720 kg·m⁻³, 6.76% and 1.47% error respectively as compared with standard techniques. Silicon, a soft material, was found by EMBE to have a bulk modulus of 1.9 GPa and density of 1380 kg·m⁻³. Error as compared to standard methods is −7.4% for the bulk modulus and −11.0%. The relatively similar density and bulk modulus values of silicone to water caused a decrease in the signal to noise ratio (SNR) during the data acquisition phase. For this work, to maintain uniformity and primarily focus on the application of the EBME technique exclusively, no additional advanced signal processing techniques were performed to increase the SNR for silicon. However, scaling the elastography logarithmically can enhance the apparent spatial resolution.

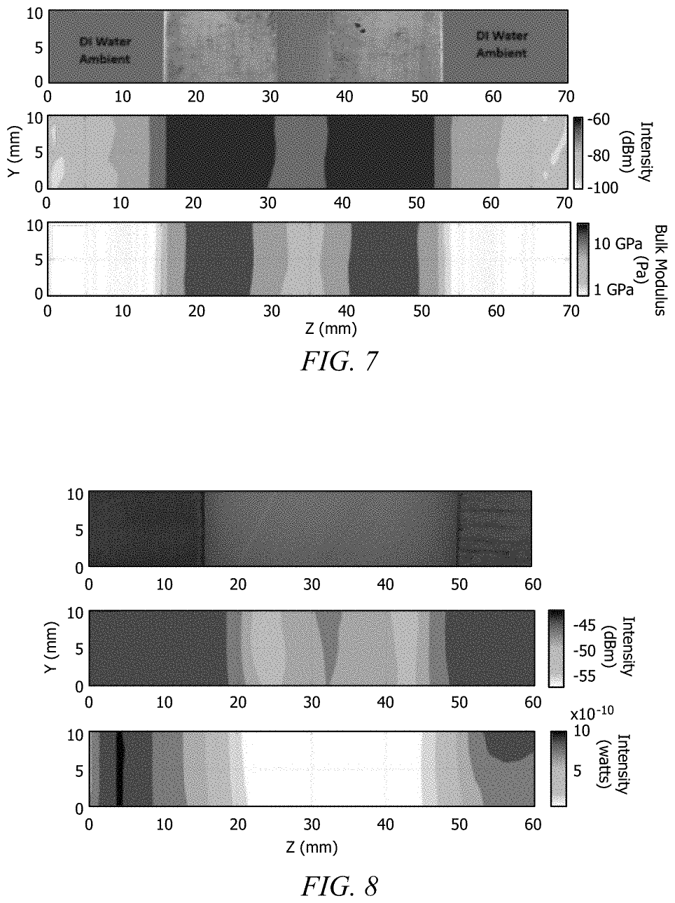

The logarithmically scaled EBME is given in FIGS. 7B and 7C, where the bulk modulus (K) is scaled as log₁₀ K. The visual impact of the gradients in effective bulk modulus are reduced by logarithmic binning. As compared to the normal A-Mode methods that map intensity, the EBME bulk modulus spatial resolution does not replicate the distinct aluminum-silicone boundaries. However, logarithmic EBME bulk modulus much more clearly delineates between different materials as the silicone in the center of the aluminum is clearly distinguishable from the ambient water. For diagnostics of hard and soft systems, this can be invaluable in determining potential defects that are undesirable versus those that are inconsequential.

### Experiment 2—Hard Material Composite Sample

In the second experiment, a hard material composite consisting of independent blocks of copper, PVC plastic, and aluminum was scanned using the same setup as Experiment 1 to evaluate the capability of EBME to distinguish between hard materials. The scanned image can be seen in FIGS. 8A, 9A, and 10A. The total scanned area was 10 mm along the vertical, y-axis at 2 mm intervals, and 60 mm along the lateral, z-axis at 1 mm intervals. The widths of each material in the sample area were non-uniform, with copper occupying 15 mm of the total width, and PVC and aluminum at 35 mm and 10 mm.

FIG. 8 gives both the linear (FIG. 8C) and logarithmic (FIG. 8B) A-Mode resultant image from the hard sample composite. The setup is not optimized for high boundary resolution, however, whereas the logarithmic scale does give a relatively accurate representation of the hard material boundaries, the intensity scale alone does not adequately distinguish between copper and aluminum on the far left and far right of FIG. 8B. The linear A-Mode method performs much worse as the boundaries are not clear, and the aluminum and copper are not distinguishable. In both cases, A-mode imaging does show the PVC material as significantly different than both aluminum and copper.

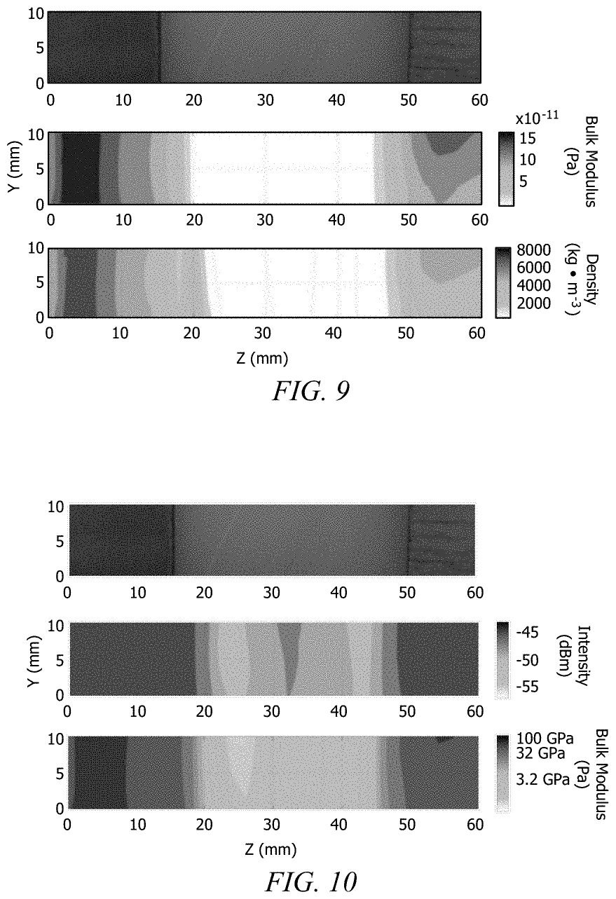

From the EBME derived bulk modulus (FIG. 9B) and density (FIG. 9C), three distinct materials and their boundaries can be readily recognized. The large contrast in elastic properties between the PVC plastic and metals allows for the estimation of its width from the linear EBME. Using the bulk modulus in FIG. 9B, PVC is 31 mm with a bulk modulus of 9.26 GPa resulting in −11% error in the width resolution and 5.5% error from a bulk modulus of 8.75 GPa determined using accepted standard techniques. For copper, the averaged bulk modulus is 138 GPa, about 4.4% off of the value derived from standard techniques. Aluminum has the highest degree of error in the bulk modulus at −10.5%, an averaged 61.4 GPa from EBME versus 68.6 GPa standardized. Density is also relatively well characterized as the EBME derived values from averaging each of the areas of the sample materials comes to 7720 kg·m⁻³, 1480 kg·m⁻³, and 2520 kg·m⁻³ for copper, PVC, and aluminum respectively. Errors for the density as compared with non-EBME techniques are −3.3%, 5.3%, and −6.0%. Additionally, as FIG. 10 shows, logarithmic scaled EBME (FIG. 10C) more accurately represents the hard material distribution shown in FIG. 10A as compared with the logarithmic A-Mode result shown in FIG. 10B.

### Experiment 3—Soft Tissue Phantom Composite

Soft materials, specifically soft materials that mimic organic tissues, present special challenges for ultrasonic characterization. The materials are commonly dispersive and attenuate sound much faster than hard materials. Additionally, tissue-like materials may have features similar to water, making them indistinguishable in standard A-Mode imaging modalities. For Experiment 3, examination of two separate samples comprised of composites of gelatin tissue phantoms was carried out. Sample 1 consisted of gelatin tissue phantoms where three (3) gelatin blocks, 22.5%, 18.0%, and 3.5% gelatin respectively, were placed adjacent to each other.

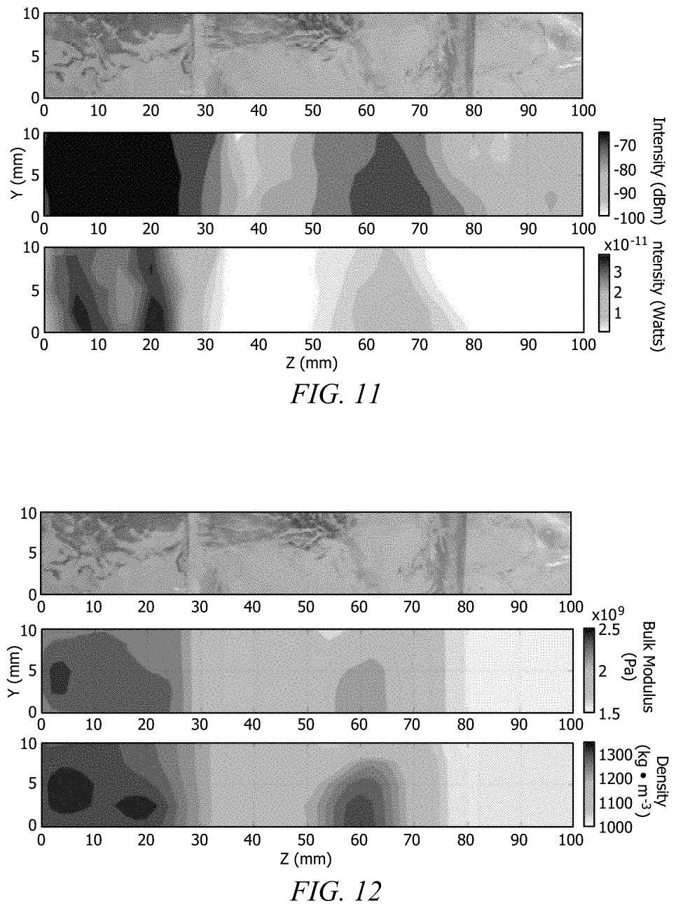

The total scanned area for Sample 1 was 100×10 mm, where the 22.5% gelatin tissue phantom was 28 mm wide, the 18.0% gelatin tissue phantom 52 mm in width, and 3.5% gelatin a width of 20 mm. The A-Mode image of Sample 1 is given in FIG. 11. In both the logarithmic (FIG. 11B) and linear (FIG. 11C) scaled images, only the boundary of the highest weight % gelatin is clearly distinguishable, with an average width of 31.5 mm between the logarithmic and linear scales. Based on comparison of the image of the sample (FIG. 11A), and the A-Mode mapping, no reasonable information is gleaned for the 18% and 3.5% gelatin samples using the A-Mode modality.

Unlike standard A-Mode, the three different tissue phantoms can clearly be distinguished by the EBME technique as seen in FIG. 12. The scanned image can be seen in FIG. 12A. From FIG. 12B, EBME shows the 22.5% gelatin to be ˜28 mm wide with a bulk modulus of 2.35 GPa. The 18.0% and 3.5% gelatin tissue phantoms are ˜51 mm and 21 mm with averaged effective bulk modulus of 2.02 GPa and 1.63 GPa. The efficacy of the EBME is supported by bulk modulus values determined using other invasive methods as 2.79 GPa, 2.33 GPa, and 1.74 GPa.

Density values for the three composites are also clearly distinct and able to be used to characterize the sample as a composite of three distinct materials (FIG. 12C). Averaged density and widths from EBME are 1271.2 kg·m⁻³ and 32 mm for 22.5% gelatin, 1183.5 kg·m⁻³ and ˜50 mm for 18.0% gelatin, and 1085.4 kg·m⁻³ and 18 mm for the lowest ratio 3.5% gelatin tissue phantom. The averaged values replicate those determined using standard means with errors of 5.2%, 1.6%, and 2.4% for each of the individual materials.

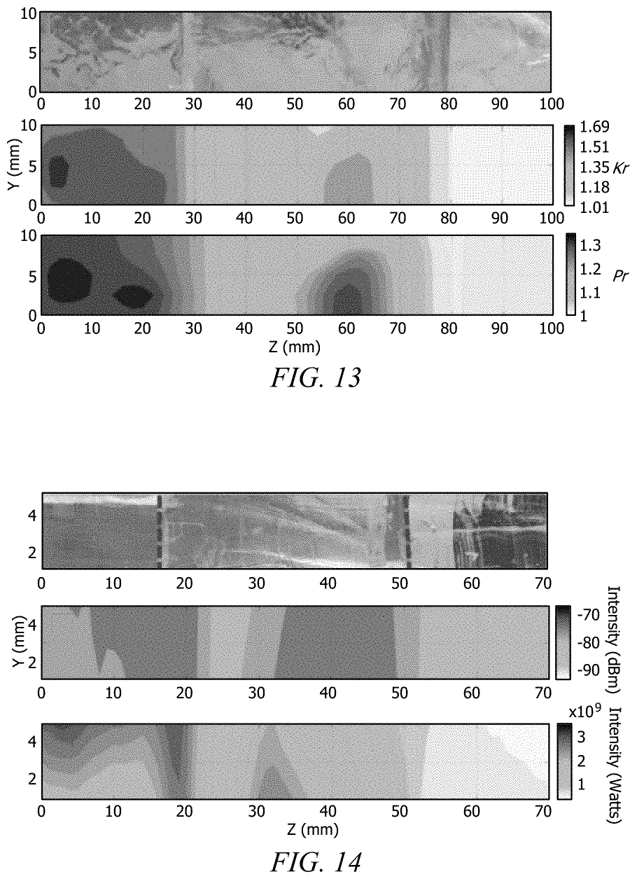

Ultrasonic images used for evaluation are commonly scaled to a standard material or medium. Here, we created a scaled parameter for the EBME determined bulk modulus and density, where the values are scaled to the ambient medium, water (FIGS. 13A-C). The relative bulk modulus, Kᵣ=Kₛₐₘₚₗₑ/K_{water}, (FIG. 13B) and the relative density,

$${ρ}_{r} = \frac{{ρ}_{sample}}{{ρ}_{w a t e r}} ,$$

(FIG. 13C) serve to indicate the extent of the deviation of an examined material from the ambient medium. All the tissue samples maintain relative values close to that of water. In practical applications, Kᵣ and ρᵣ could be calibrated to an ideal sample where Kᵣ=Kₛₐₘₚₗₑ/K_{Ideal} and ρᵣ=ρₛₐₘₚₗₑ/ρ_{Ideal}, which might be much easier to use. Unlike the standard A-Mode case, the relative values for the samples allow for the 3.5% tissue phantom to be visualized with an apt comparison to water. In medical applications, scaling to healthy tissues values may lead to faster recognition of potentially harmful artifacts in tissue.

For Sample 2, we applied the same techniques to tissue phantoms with compositions that were more similar than Sample 1. Three tissue phantoms of 16.8%, 10.0%, and 6.8% gelatin were synthesized. The widths of the phantoms were 16 mm, 35 mm, and 19 mm, with densities from standard techniques of 1163 kg·m⁻³, 1106 kg·m⁻³, and 1064 kg·m⁻³, and bulk modulus values of 2.31 GPa, 2.13 GPa, and 2.01 GPa. The low variation in physical properties amongst the phantoms was selected to mimic low variation experienced in tissues in practice.

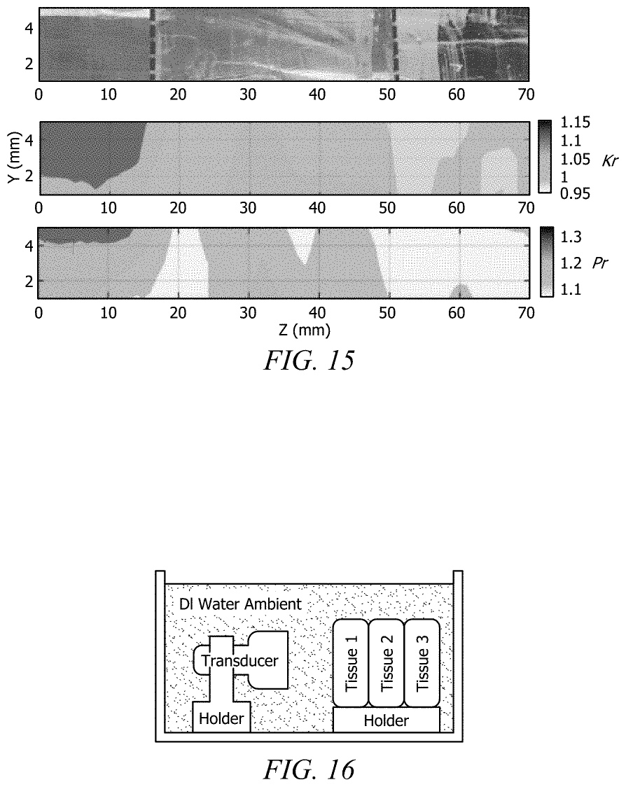

The reference A-Mode scan of Sample 2 is given in FIGS. 14A-C. Both the logarithmic and linear scale figures identify the existence of the lowest concentration tissue phantom as relatively homogenous material on the right (FIGS. 14B, 14C). However, the rest of Sample 2 is not clearly characterized when compared to the reference, FIG. 15A. FIGS. 15B and 15C both visually indicate composites of more than three (3) materials, or strong inhomogeneity in the tissue phantoms.

The relative bulk modulus and density of Sample 2 is shown in FIG. 15 with much greater clarity than standard A-Mode imaging. Visually, Kᵣ most strongly indicates the existence of three distinct materials in the sample (FIG. 15B). The widths of the samples using EBME with scaled elastic values is 18 mm for the 16.5% phantom on the left, 30 mm for the 10.0% phantom in the center, and 20 mm for the lowest gelatin concentration material on the far right of FIG. 15A. The estimated widths are 12.5%, −14.3%, and 5.3% off the actual values, but still vastly superior to the A-Mode technique which could not identify three clear materials.

For Sample 2, weak reflection from the tissue phantoms and the lack of application of advanced signal processing techniques led to relatively high SNR as compared with hard materials such as steel. The impact of the low SNR was most strongly manifested in the EBME derived average density and bulk modulus values for the samples which had errors of −10.0%, −12.7%, and −43.3% for the bulk modulus, and −4.3%, −1.3%, and −40.6% for density. The highest degree of error was from the tissue was independently measure acoustic impedance very close to water, which caused a greatly reduced reflected signal to process using EBME. Regardless, without advanced signal processing techniques, the EBME method greatly improved the clarity of the samples as compared with standard A-Mode methods while being effective with remote application.

### Experiment 4—Soft Materials Arranged Axially

Experiments 1-3 all scanned the samples laterally, where each material was axially uniform. However, practical application of EBME will require that the technique can be applied to samples comprise of an unknown number of composite materials. The generalized application of EBME to n-materials was derived in the first section of the work with Eqs. 4-6 describing the application to an arbitrary number of materials. Experiment 3 explored the capability of EBME to distinguish soft materials of similar elastic values, though the material arrangement was lateral. For Experiment 4, we examine EBME for an axial arrangement of three (3) tissue phantoms. The experimental setup of EBME applied to three layers tissue phantoms with different bulk modulus and density arranged axially is shown in FIG. 16.

The three tissue phantoms were synthesized following the same procedures as above with the various gelation ratios. The density each of the phantoms in Experiment 4 was 1208.2 kg m⁻³, 1164.6 kg·m⁻³, and 1059.5 kg·m⁻³, with corresponding bulk modulus of 2.794 GPa, 2.328 GPa, and 1.744 GPa. Eight experiments were performed in which the order of the tissues was varied and the EBME derived bulk modulus and density determined using Eqs. 4, 9 and 10. The resultant values were averaged for the results.

EBME performed well with the bulk modulus of the phantoms 2.65±0.08 GPa, 2.29±0.14 GPa, 1.88±0.10 GPa for errors of 5.37%, 1.69%, and 7.72%. It should be noted, that consistent with the procedures of this work, no advanced signal processing techniques were used to improve the SNR and potentially reduce errors in the determined values. The density, as determined axially with the material arrangement varied, came to 1245±40 kg·m⁻³, 1135±56 kg·m⁻³, 979±116 kg·m⁻³. The values deviated from standard tests by 3.10%, 2.52%, and 7.59% showing EBME as a very effective, non-destructive method for remotely performing elastography.

Having described various systems and methods herein, certain embodiments can include, but are not limited to:

In a first embodiment, a method of non-destructive evaluation of mechanical properties of a material using ultrasonic waves in a monostatic configuration comprises remotely scanning a sample of the material without directly contacting the sample, measuring an acoustic impedance of the scanned sample, and calculating mechanical properties of the material using the acoustic impedance.

A second embodiment can include the method of the first embodiment, wherein the scanning is performed using strain map imaging and shear wave elastography.

A third embodiment can include the method of the second embodiment, wherein the scanning comprises emitting from a transducer a plurality of longitudinal and/or transverse transmitted pulses towards the sample without direct application of external radiational stress or cyclidic stress on the sample, and receiving at the transducer a plurality of reflected pulses.

A fourth embodiment can include the method of the third embodiments, wherein the transducer also transmits and receives transverse pulses.

A fifth embodiment can include the method of the third embodiment, wherein the transducer and the sample are disposed in an ambient medium, wherein the pressure-voltage sensitivity of the transducer is standardized to the ambient medium, and wherein the transmitted pulses travel from the transducer through the ambient medium to the sample.

A sixth embodiment can include the method of the fifth embodiment, wherein the ambient medium consists of DI water.

A seventh embodiment can include the method of the fifth embodiment, wherein the sample is mounted within the ambient medium on a Y-axis/Z-axis translation stage connected to a controller, and wherein the transducer is an ultrasonic pulser/receiver connected to a 0.5 MHz unfocused immersion transducer component.

An eighth embodiment can include the method of the fifth embodiment, wherein the acoustic impedance of the scanned sample is measured by inputting transducer signals from the transmitted pulses and the reflected pulses into an oscilloscope connected to a computer, the computer having a configuration and programming instructions for processing acoustic impedance using Formula (8),

$$Z = {( \frac{{Z}_{0}^{2} {\int }_{{t}_{i}}^{{r}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3} , \qquad ( 8 )$$

where S is pressure-voltage sensitivity coefficient of the detector in units of

$$\frac{P a}{V} ,$$

where τ is the time delay between the starting point of a first emitted pulse and a first reflected pulse, where α is a scaling coefficient ranging from 1 to 2, wherein α approaches 2 in soft materials including tissue, and wherein α approaches 1 for hard materials including metal, where t_{f}, is an end of a pulse envelope, where tᵢ is set as the point when a continuous 0.05 μs [pulse] exceeds 110% of a maximum noise level, where Σ_{k=f}_{₁}^{N}Yₙ(f)ₖ is intensity of the n^{th} reflected pulse, where Vₒ(t) is time dependent pulse voltage amplitude at 0, and where Vₑ(t) is time dependent pulse voltage amplitude emitted.

A ninth embodiment can include the method of the eighth embodiment, wherein the mechanical properties comprise EBME density and EBME bulk modulus, the computer having a configuration and programming instructions for processing EBME density and EBME bulk modulus from acoustic impedance, where EBME density is obtained using Formula (9) with L wave mode, zero external force applied, ρ density values, in ambient medium, with input values d, S, Z₀

$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 9 )$$

where EBME bulk modulus is obtained using Formula (10) with L wave mode, zero external force applied, K Elasticity values, in ambient medium, with input values d, S, Z₀

$$K = {c ( \frac{{Z}_{0}^{2} {\int }_{{t}_{i}}^{{t}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3} \qquad ( 10 )$$

where d is sample thickness, where S is transducer sensitivity coefficient, and where Z₀ is acoustic impedance of the ambient medium.

A tenth embodiment can include the method of the eighth embodiment, wherein scaling coefficient α is selected as equivalent to 6.8% gelatin tissue phantom (liver tissue), 10% gelatin tissue phantom (tumor stage 1), and 16.8% gelatin tissue phantom (tumor stage 2).

An eleventh embodiment can include the method of the eighth embodiment, wherein scaling coefficient α is selected at 1 for a hard material.

A twelfth embodiment can include the method of the eighth embodiment, wherein scaling coefficient α is selected at between 1.4 and 1.8 for a composite material.

A thirteenth embodiment can include the method of the eighth embodiment, wherein the transducer signals are processed in a digital signal processor to increase the signal-to-noise ratio (SNR) of the transducer signals before processing by the oscillator connected to the computer.

A fourteenth embodiment can include the method of the eighth embodiment, wherein the computer programming instructions include instructions for recording waveform and frequency spectrum for 20 s at multiple scan locations at 2 mm intervals on the y-axis and 1 mm intervals on the vertical, z-axis.

In a fifteenth embodiment, a system for non-destructive evaluation of mechanical properties of a material using ultrasonic waves in a monostatic configuration comprising a transducer configured to remotely scan a sample of the material without directly contacting the sample, and a computer having a configuration and programming instructions to measure an acoustic impedance of the scanned sample and calculate mechanical properties of the material using the acoustic impedance.

A sixteenth embodiment can include the system of the fifteenth embodiment, wherein the scan is performed using strain map imaging and shear wave elastography.

A seventieth embodiment can include the system of the fifteenth embodiment, wherein the transducer is mounted on a holder, and wherein the holder is mounted within a container filled with an ambient medium.

An eighteenth embodiment can include the system of the seventeenth embodiment, further comprising: a 2D translation stage controller connected to the computer, and a Y-axis/Z-axis translation stage connected to the controller, the translation stage having a sample holding element for holding a sample within the ambient medium.

A nineteenth embodiment can include the system of the fifteenth embodiment, further comprising two connected stepper motor translation stages configured to move along the lateral (y-) and vertical (z-) axes relative to the sample using a Universal Motion Controller/Driver.

A twentieth embodiment can include the system of the nineteenth embodiment, wherein the computer programming instructions include instructions for recording waveform and frequency spectrum for 20 s at multiple scan locations at 2 mm intervals on the y-axis and 1 mm intervals on the vertical, z-axis.

A twenty-first embodiment can include the system of the fifteenth embodiment, wherein the transducer is a 10V+ negative spike excitation pulser/receiver connected to a 0.5 MHz unfocused immersion transducer.

A twenty-second embodiment can include the system of the fifteenth embodiment, wherein the transducer is configured to emit a plurality of longitudinal and/or transverse transmitted pulses towards the sample without direct application of external radiational stress or cyclidic stress on the sample, and receive a plurality of reflected pulses.

A twenty-third embodiment can include the system of the twenty-second embodiment, wherein the transducer and the sample are disposed in an ambient medium, wherein the pressure-voltage sensitivity of the transducer is standardized to the ambient medium, and wherein the transmitted pulses travel from the transducer through the ambient medium to the sample.

A twenty-fourth embodiment can include the system of the twenty-third embodiment, wherein the ambient medium consists of DI water.

A twenty-fifth embodiment can include the system of the fifteenth embodiment, further comprising: a pulse generator/receiver unit connected to the transducer, and an oscilloscope connected to the pulse generator/receiver unit, wherein the computer is connected to the oscilloscope.

The twenty-sixth embodiment can include the system of the twenty-fifth embodiment, wherein the oscilloscope is a mixed domain oscilloscope with 2-4 analog channels, 100-1000 MHz integrated spectrum analyzer.

The twenty-seventh embodiment can include the system of the twenty-fifth embodiment, wherein the acoustic impedance of the scanned sample is measured by inputting transducer signals from the transmitted pulses and the reflected pulses into the oscilloscope, wherein the computer programming instructions include instructions for processing acoustic impedance using Formula (8),

$$Z = {( \frac{{Z}_{0}^{2} {\int }_{{t}_{i}}^{{r}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3} , \qquad ( 8 )$$

where S is pressure-voltage sensitivity coefficient of the detector in units of

$$\frac{P a}{V} ,$$

where τ is the time delay between the starting point of a first emitted pulse and a first reflected pulse, where α is a scaling coefficient ranging from 1 to 2, wherein α approaches 2 in soft materials including tissue, and wherein α approaches 1 for hard materials including metal, where t_{f}, is an end of a pulse envelope, where tᵢ is set as the point when a continuous 0.05 μs [pulse] exceeds 110% of a maximum noise level, where Σ_{k=f}_{₁}^{N}Yₙ(f)ₖ is intensity of the n^{th} reflected pulse, where Vₒ(t) is time dependent pulse voltage amplitude at 0, and where Vₑ(t) is time dependent pulse voltage amplitude emitted.

The twenty-eighth embodiment can include the system of the twenty-seventh embodiment, wherein the mechanical properties comprise EBME density and EBME bulk modulus, wherein the computer programming instructions include instructions for processing EBME density and EBME bulk modulus from acoustic impedance, where EBME density is obtained using Formula (9) with L wave mode, zero external force applied, ρ density values, in ambient medium, with input values d, S, Z₀

$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 9 )$$

where EBME bulk modulus is obtained using Formula (10) with L wave mode, zero external force applied, K Elasticity values, in ambient medium, with input values d, S, Z₀

$$K = {c ( \frac{{Z}_{0}^{2} {\int }_{{t}_{i}}^{{t}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3} \qquad ( 10 )$$

where d is sample thickness, where S is transducer sensitivity coefficient, and where Z₀ is acoustic impedance of the ambient medium.

In a twenty ninth embodiment, a process for non-destructive evaluation of the mechanical properties of a material using ultrasonic waves in a monostatic configuration, comprises: (i) remotely scanning a sample of the material [using strain map imaging and shear wave elastography], wherein scanning is emitting from a transducer a plurality of longitudinal and/or transverse transmitted pulses towards the sample without direct application of external radiational stress or cyclidic stress on the sample, and receiving at the transducer a plurality of reflected pulses, wherein the transducer and the sample are disposed in an ambient medium, wherein the pressure-voltage sensitivity of the transducer is standardized to the ambient medium, and wherein the transmitted pulses travel from the transducer through the ambient medium to the sample, (ii) measuring an acoustic impedance of the scanned sample by inputting transducer signals from the transmitted pulses and the reflected pulses into an oscilloscope connected to a computer, the computer having a configuration and programming instructions for processing acoustic impedance using Formula (8),

$$Z = {( \frac{{Z}_{0}^{2} {\int }_{{t}_{i}}^{{r}_{f}} {( {V}_{e} ( t ) \cdot S - {V}_{0} ( t + τ ) \cdot S )}^{2} dt}{{α}^{2} ( {t}_{f} - {t}_{i} ) {\sum }_{k = {f}_{1}}^{N} {Y ( f )}_{k}} )}^{- 3}$$

where S is pressure-voltage sensitivity coefficient of the detector in units of

$$\frac{Pa}{V} ,$$

where τ is the time delay between the starting point of a first emitted pulse and a first reflected pulse, where α is a scaling coefficient ranging from 1 to 2, wherein α approaches 2 in soft materials including tissue, and wherein α approaches 1 for hard materials including metal, where t_{f}, is an end of a pulse envelope, where tᵢ is set as the point when a continuous 0.05 μs [pulse] exceeds 110% of a maximum noise level, where Σ_{k=f}_{₁}^{N}Yₙ(f)ₖ is intensity of the n^{th} reflected pulse, where Vₒ(t) is time dependent pulse voltage amplitude at 0, where Vₑ(t) is time dependent pulse voltage amplitude emitted, and (iii) calculating mechanical properties of the material using the acoustic impedance, wherein the mechanical properties are EBME density and EBME bulk modulus, the computer having a configuration and programming instructions for processing EBME density and EBME bulk modulus from acoustic impedance, where EBME density is obtained using Formula (9) with L wave mode, zero external force applied, ρ density values, in ambient medium, with input values d, S, Z₀

$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3}$$

where EBME bulk modulus is obtained using Formula (10) with L wave mode, zero external force applied, K Elasticity values, in ambient medium, with input values d, S, Z₀

$$K = {c ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 10 )$$

where d is sample thickness, where S is transducer sensitivity coefficient, and where Z₀ is acoustic impedance of the ambient medium.

A thirtieth embodiment can include the process of the twenty ninth embodiment, wherein scaling coefficient α is selected as equivalent to 6.8% gelatin tissue phantom (liver tissue), 10% gelatin tissue phantom (tumor stage 1), and 16.8% gelatin tissue phantom (tumor stage 2).

A thirty first embodiment can include the process of the twenty ninth embodiment, wherein scaling coefficient α is selected at 1 for a hard material.

A thirty second embodiment can include the process of the twenty ninth embodiment, wherein scaling coefficient α is selected at between 1.4 and 1.8 for a composite material.

A thirty third embodiment can include the process of the twenty ninth embodiment, further comprising where the transducer signals are processed in a digital signal processor to increase the signal-to-noise ratio (SNR) of the transducer signals before processing by the oscillator connected to the computer.

A thirty fourth embodiment can include the process of the twenty ninth embodiment, wherein the transducer also transmits and receives transverse pulses.

A thirty fifth embodiment can include the process of the twenty ninth embodiment, wherein the computer programming instructions include instructions for recording waveform and frequency spectrum for 20 s at multiple scan locations at 2 mm intervals on the y-axis and 1 mm intervals on the vertical, z-axis.

A thirty sixth embodiment can include the process of the twenty ninth embodiment, wherein the ambient medium consists of DI water.

A thirty seventh embodiment can include the process of the twenty ninth embodiment, wherein the sample is mounted within the ambient medium on a Y-axis/Z-axis translation stage connected to a controller, wherein the transducer is an ultrasonic pulser/receiver connected to a 0.5 MHz unfocused immersion transducer component.

In a thirty eighth embodiment, an apparatus for non-destructive evaluation of the mechanical properties of a material using ultrasonic waves in a monostatic configuration, comprises: (a) an immersion transducer mounted on a holder, the holder mounted within a container filled with an ambient medium; (b) a pulse generator/receiver unit connected to the transducer; (c) an oscilloscope connected to the pulse generator/receiver unit, (d) a computer connected to the oscilloscope, (e) a 2D translation stage controller connected to the computer, and (f) a Y-axis/Z-axis translation stage connected to the controller, the translation stage having a sample holding element for holding a sample within the ambient medium, wherein the apparatus is configured to (i) remotely scan a sample of the material [using strain map imaging and shear wave elastography], wherein scanning is emitting from a transducer a plurality of longitudinal transmitted pulses towards the sample without direct application of external radiational stress or cyclidic stress on the sample, and receiving at the transducer a plurality of reflected pulses, wherein the transducer and the sample are disposed in an ambient medium, wherein the pressure-voltage sensitivity of the transducer is standardized to the ambient medium, and wherein the transmitted pulses travel from the transducer through the ambient medium to the sample, (ii) measure an acoustic impedance of the scanned sample by inputting transducer signals from the transmitted pulses and the reflected pulses into an oscillator connected to a computer, the computer having a configuration and programming instructions for processing acoustic impedance using Formula (8),

$$Z = {( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} , \qquad ( 8 )$$

where S is pressure-voltage sensitivity coefficient of the detector in units of

$$\frac{Pa}{V} ,$$

where τ is the time delay between the starting point of a first emitted pulse and a first reflected pulse, where α is a scaling coefficient ranging from 1 to 2, wherein α approaches 2 in soft materials including tissue, and wherein α approaches 1 for hard materials including metal, where t_{f}, is an end of a pulse envelope, where tᵢ is set as the point when a continuous 0.05 μs [pulse] exceeds 110% of a maximum noise level, where Σ_{k=f}_{₁}^{N}Yₙ(f)ₖ is intensity of the n^{th} reflected pulse, where Vₒ(t) is time dependent pulse voltage amplitude at 0, where Vₑ(t) is time dependent pulse voltage amplitude emitted, and (iii) calculate mechanical properties of the material using the acoustic impedance, wherein the mechanical properties are EBME density and EBME bulk modulus, the computer having a configuration and programming instructions for processing EBME density and EBME bulk modulus from acoustic impedance, where EBME density is obtained using Formula (9) with L wave mode, zero external force applied, ρ Elasticity values, in ambient medium, with input values d, S, Z₀

$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 9 )$$

where EBME bulk modulus is obtained using Formula (10) with L wave mode, zero external force applied, K Elasticity values, in ambient medium, with input values d, S, Z₀

$$K = {c ( \frac{{Z}_{0}^{2} {\int }_{{t}_{i}}^{{t}_{f}} {( {V}_{e} ( t ) S - {V}_{0} ( t + τ ) \cdot S )}^{2} d t}{{a}^{2} ( {t}_{f} - {t}_{i} ) {\sum }_{k = {f}_{1}}^{N} {Y ( f )}_{k}} )}^{- 3} \qquad ( 10 )$$

where d is sample thickness, where S is transducer sensitivity coefficient, and where Z₀ is acoustic impedance of the ambient medium.

A thirty ninth embodiment can include the apparatus of the thirty eighth embodiment, wherein the stepper motor translation stages comprise two connected stepper motor translation stages configured to move along the lateral (y-) and vertical (z-) axes relative to the sample using a Universal Motion Controller/Driver.

A fortieth embodiment can include the apparatus of the thirty eighth embodiment, wherein the transducer is a 10V+ negative spike excitation pulser/receiver connected to a 0.5 MHz unfocused immersion transducer.

A forty first embodiment can include the apparatus of the thirty eighth embodiment, wherein the oscilloscope is a mixed domain oscilloscope with 2-4 analog channels, 100-1000 MHz integrated spectrum analyzer.

A forty second embodiment can include the apparatus of the thirty eighth embodiment, wherein the computer programming instructions include instructions for recording waveform and frequency spectrum for 20 s at multiple scan locations at 2 mm intervals on the y-axis and 1 mm intervals on the vertical, z-axis.

A forty third embodiment can include the apparatus of the thirty eighth embodiment, wherein the ambient medium consists of DI water.

Embodiments are discussed herein with reference to the Figures. However, those skilled in the art will readily appreciate that the detailed description given herein with respect to these figures is for explanatory purposes as the systems and methods extend beyond these limited embodiments. For example, it should be appreciated that those skilled in the art will, in light of the teachings of the present description, recognize a multiplicity of alternate and suitable approaches, depending upon the needs of the particular application, to implement the functionality of any given detail described herein, beyond the particular implementation choices in the following embodiments described and shown. That is, there are numerous modifications and variations that are too numerous to be listed but that all fit within the scope of the present description. Also, singular words should be read as plural and vice versa and masculine as feminine and vice versa, where appropriate, and alternative embodiments do not necessarily imply that the two are mutually exclusive.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although claims may be formulated in this application or of any further application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicant(s) hereby give notice that new claims may be formulated to such features and/or combinations of such features during the prosecution of the present Application or of any further Application derived therefrom.

1. A method of non-destructive evaluation of mechanical properties of a material using ultrasonic waves in a monostatic configuration, the method comprising:
remotely scanning a sample of the material without directly contacting the sample, wherein the scanning comprises emitting from a transducer a plurality of longitudinal and/or transverse transmitted pulses towards the sample without direct application of external radiational stress or cyclidic stress on the sample, and receiving at the transducer a plurality of reflected pulses, and wherein the transducer and the sample are disposed in an ambient medium;
measuring an acoustic impedance of the scanned sample; and
calculating mechanical properties of the material using the acoustic impedance.

2. The method of claim 1, wherein the scanning is performed using strain map imaging and shear wave elastography.

3. (canceled)

4. The method of claim 1, wherein the transducer also transmits and receives transverse pulses.

5. The method of claim 1, wherein the pressure-voltage sensitivity of the transducer is standardized to the ambient medium, and wherein the transmitted pulses travel from the transducer through the ambient medium to the sample.

6. (canceled)

7. The method of claim 5, wherein the ambient medium consists of water, wherein the sample is mounted within the ambient medium on a Y-axis/Z-axis translation stage connected to a controller, and wherein the transducer is an ultrasonic pulser/receiver connected to a 0.5 MHz unfocused immersion transducer component.

8. The method of claim 5, wherein the acoustic impedance of the scanned sample is measured by inputting transducer signals from the transmitted pulses and the reflected pulses into an oscilloscope connected to a computer, the computer having a configuration and programming instructions for processing acoustic impedance using Formula (8),
$$Z = {( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} , \qquad ( 8 )$$
where S is pressure-voltage sensitivity coefficient of the detector in units of
$$\frac{Pa}{V} ,$$
where τ is the time delay between the starting point of a first emitted pulse and a first reflected pulse,
where α is a scaling coefficient ranging from 1 to 2, wherein α approaches 2 in soft materials including tissue, and wherein α approaches 1 for hard materials including metal,
where t_{f}, is an end of a pulse envelope,
where tᵢ is set as the point when a continuous 0.05 μs [pulse] exceeds 110% of a maximum noise level,
where Σ_{k=f}_{₁}^{N}Yₙ(f)ₖ is intensity of the n^{th} reflected pulse,
where Vₒ(t) is time dependent pulse voltage amplitude at 0, and
where Vₑ(t) is time dependent pulse voltage amplitude emitted.

9. The method of claim 8, wherein the mechanical properties comprise EBME density and EBME bulk modulus, the computer having a configuration and programming instructions for processing EBME density and EBME bulk modulus from acoustic impedance,
where EBME density is obtained using Formula (9) with L wave mode, zero external force applied, ρ density values, in ambient medium, with input values d, S, Z₀
$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 9 )$$
where EBME bulk modulus is obtained using Formula (10) with L wave mode, zero external force applied, K Elasticity values, in ambient medium, with input values d, S, Z₀
$$K = {c ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 10 )$$
where d is sample thickness,
where S is transducer sensitivity coefficient, and
where Z₀ is acoustic impedance of the ambient medium.

10. The method of claim 8, wherein scaling coefficient α is selected as equivalent to 6.8% gelatin tissue phantom (liver tissue), 10% gelatin tissue phantom (tumor stage 1), and 16.8% gelatin tissue phantom (tumor stage 2).

11. The method of claim 8, wherein scaling coefficient α is selected at 1 for a hard material, or wherein scaling coefficient α is selected at between 1.4 and 1.8 for a composite material.

12. (canceled)

13. The method of claim 8, wherein the transducer signals are processed in a digital signal processor to increase the signal-to-noise ratio (SNR) of the transducer signals before processing by the oscillator connected to the computer.

14. (canceled)

15. A system for non-destructive evaluation of mechanical properties of a material using ultrasonic waves in a monostatic configuration, the system comprising:
a transducer configured to remotely scan a sample of the material without directly contacting the sample, wherein the transducer is configured to emit a plurality of longitudinal and/or transverse transmitted pulses towards the sample without direct application of external radiational stress or cyclidic stress on the sample, and receive a plurality of reflected pulses, and wherein the transducer and the sample are disposed in an ambient medium; and
a computer having a configuration and programming instructions to measure an acoustic impedance of the scanned sample and calculate mechanical properties of the material using the acoustic impedance.

16. The system of claim 15, wherein the scan is performed using strain map imaging and shear wave elastography.

17. (canceled)

18. (canceled)

19. The system of claim 15, further comprising two connected stepper motor translation stages configured to move along the lateral (y-) and vertical (z-) axes relative to the sample using a Universal Motion Controller/Driver, and wherein the computer programming instructions include instructions for recording waveform and frequency spectrum for 20 s at multiple scan location at 2 mm intervals on the y-axis and 1 mm intervals on the vertical, z-axis.

20. (canceled)

21. The system of claim 15, wherein the transducer is a 10V+ negative spike excitation pulser/receiver connected to a 0.5 MHz unfocused immersion transducer.

22. (canceled)

23. The system of claim 15, wherein the pressure-voltage sensitivity of the transducer is standardized to the ambient medium, wherein the transmitted pulses travel from the transducer through the ambient medium to the sample, and wherein the ambient medium consists of DI water.

24. (canceled)

25. The system of claim 15, further comprising:
a pulse generator/receiver unit connected to the transducer; and
an oscilloscope connected to the pulse generator/receiver unit, wherein the computer is connected to the oscilloscope.

26. (canceled)

27. The system of claim 25, wherein the acoustic impedance of the scanned sample is measured by inputting transducer signals from the transmitted pulses and the reflected pulses into the oscilloscope, wherein the computer programming instructions include instructions for processing acoustic impedance using Formula (8),
$$Z = {( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} . \qquad ( 8 )$$
where S is pressure-voltage sensitivity coefficient of the detector in units of
$$\frac{Pa}{V} ,$$
where τ is the time delay between the starting point of a first emitted pulse and a first reflected pulse,
where α is a scaling coefficient ranging from 1 to 2, wherein α approaches 2 in soft materials including tissue, and wherein α approaches 1 for hard materials including metal,
where t_{f}, is an end of a pulse envelope,
where tᵢ is set as the point when a continuous 0.05 μs [pulse] exceeds 110% of a maximum noise level,
where Σ_{k=f}_{₁}^{N}Yₙ(f)ₖ is intensity of the n^{th} reflected pulse,
where Vₒ(t) is time dependent pulse voltage amplitude at 0, and
where Vₑ(t) is time dependent pulse voltage amplitude emitted.

28. The system of claim 27, wherein the mechanical properties comprise EBME density and EBME bulk modulus, wherein the computer programming instructions include instructions for processing EBME density and EBME bulk modulus from acoustic impedance,
where EBME density is obtained using Formula (9) with L wave mode, zero external force applied, ρ density values, in ambient medium, with input values d, S, Z₀
$$ρ = {{c}^{- 1} ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 9 )$$
where EBME bulk modulus is obtained using Formula (10) with L wave mode, zero external force applied, K Elasticity values, in ambient medium, with input values d, S, Z₀
$$K = {c ( \frac{{Z}_{0}^{2} {\int }_{{t}_{i}}^{{t}_{f}} {( {V}_{e} ( t ) \cdot S - {V}_{0} ( t + τ ) \cdot S )}^{2} d t}{{α}^{2} ( {t}_{f} - {t}_{i} ) {\sum }_{k = {f}_{1}}^{N} {Y ( f )}_{k}} )}^{- 3} \qquad ( 10 )$$
where d is sample thickness,
where S is transducer sensitivity coefficient, and
where Z₀ is acoustic impedance of the ambient medium.

29. A system for non-destructive evaluation of mechanical properties of a material using ultrasonic waves in a monostatic configuration, the system comprising:
a transducer configured to remotely scan a sample of the material without directly contacting the sample, wherein the transducer is mounted on a holder, and wherein the holder is mounted within a container filled with an ambient medium; and
a computer having a configuration and programming instructions to measure an acoustic impedance of the scanned sample and calculate mechanical properties of the material using the acoustic impedance.

30. The system of claim 29, further comprising:
a 2D translation stage controller connected to the computer; and
a Y-axis/Z-axis translation stage connected to the controller, the translation stage having a sample holding element for holding a sample within the ambient medium.
